(19) United States
(12) Patent Application Publication
(10) Pub. No.: US 2005/0054942 A1
(43) Pub. Date: Mar. 10, 2005

(54) System and method for therapeutic drug monitoring

(76) Inventors: Richard J. Melker, Gainesville, FL (US); James Chris Sackellares, Gainesville, FL (US); Mark S. Gold, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/788,501

(22) Filed: Feb. 26, 2004

Related U.S. Application Data
(63) Continuation-in-part of application No. 10/178,877, filed on Jun. 24, 2002.
Application No. 10/178,877 is a continuation-in-part of application No. 10/054,619, filed on Jan. 22, 2002.

(51) Int. Cl. A61B 5/08; A61M 37/00

(52) U.S. Cl. 600/532; 604/19

(57) ABSTRACT

The present invention includes systems and methods for monitoring therapeutic drug concentration in blood by detecting markers, such as odors, upon exhalation by a patient after the drug is taken, wherein such markers result either directly from the drug itself or from an additive combined with the drug. In the case of olfactory markers, the invention preferably utilizes electronic sensor technology, such as the commercial devices referred to as “artificial” or “electronic” noses or tongues, to non-invasively monitor drug levels in blood. The invention further includes a reporting system capable of tracking drug concentrations in blood (remote or proximate locations) and providing the necessary alerts with regarding to ineffective or toxic drug dosages in a patient.

### CROSS-REFERENCE TO A RELATED APPLICATION

This application is a continuation-in-part of co-pending U.S. patent application Ser. No. 10/178,877, filed Jun. 24, 2002, which is a continuation-in-part of co-pending U.S. patent application Ser. No. 10/054,619, filed Jan. 22, 2002.

### FIELD OF INVENTION

The present invention relates to non-invasive monitoring of substance/compound concentrations in blood; and more particularly, to a system and method for the detection of drug concentrations in blood utilizing a breath detection system.

### BACKGROUND INFORMATION

The concentration of a drug in a patient's body is generally regulated both by the amount of drug ingested by the patient over a given time period, or the dosing regimen, and the rate at which the drug is metabolized and eliminated by the body. The drug can generally be eliminated in two different ways, depending on the chemical structure of the drug. First the drug can be chemically modified into an inactive component(s) that is then excreted. Alternatively, the drug can be excreted from the body in a substantially unadulterated form.

Historically, pharmaceutical compositions were delivered to patients according to standard doses based on the patient's weight. In the early 1970s, it was discovered with epileptic patients that pharmaceutical treatment with dosages adjusted according to blood concentration of the drug was far more efficient and demonstrated better seizure control and few side effects than with dosages adjusted according to patient weight.

It is now generally accepted that with many medications, it is necessary to monitor the concentration level of a drug in the blood stream in order to ensure optimal, therapeutic drug effect. Certain medications are ineffective if blood concentration levels are too low. Moreover, certain medications are toxic to the body when concentration levels in the blood are too high. It would also be valuable to have a means for monitoring drug concentration in blood for medications that do not require constant monitoring. By monitoring blood serum drug levels, medication dosage can be individualized within a therapeutically effective range.

For example, tricyclic or tetracyclic antidepressants (TCAs) require constant monitoring in patient blood. TCAs work by inhibiting serotonin and norepinephrine reuptake into the synaptic cleft. This group includes among its members the tricyclics imipramine, nortriptyline, and clomipramine, and the tetracyclics maprotiline and amoxapine. It is the inhibition of norepinephrine reuptake that is believed to cause TCAs side effects, which include sedation, manic episodes, profuse sweating, palpitations, increased blood pressure, tachycardia, twitches and tremors of the tongue or upper extremities, and weight gain. Compared with serotonin reuptake inhibitors (SSRIs) which are currently available, TCAs have very significant side effects, some virtually life threatening, and others merely difficult for patients to tolerate.

Although SSRIs are not more effective, and may actually be slightly less effective than some TCAs, TCAs are less attractive because they are more toxic than SSRIs and pose a greater threat of overdose. A TCA overdose results in central nervous system and cardiovascular toxicity making the relative risk of death by overdose with a TCA 2.5 to 8.5 times that with commercially available SSRI—Prozac. The greater danger with TCA is that side effects, as well as constant blood sampling, will persuade the patient not to continue treatment. Studies indicate that patients taking a classical antidepressant (TCA or MAOI) are three times as likely to drop out of treatment due to side effects and constant monitoring as patients taking Prozac.

Thus, therapeutically effective medications that require monitoring of blood serum drug levels are less likely to be prescribed by physicians in view of inconvenience in constant blood sampling and lack of patient compliance. Further, in the present era of cost-effective healthcare, considerations of prescription costs have become the primary issue for all aspects of laboratory operation. Individualization of drug therapy contributes to cost-effective patient management through detection and elimination of drug side effects; detection of unusual metabolism and adjustment of dosage based on individual metabolism; and detection of unusual metabolism and adjustment of dosage based on the effects on disease.

Drug level testing is especially important in patients being administered medications where the margin of safety between therapeutic effectiveness and toxicity is narrow. Drugs such as procainamide or digoxin, which are used to treat arhthymia; dilantin or valproic acid, which are used to treat seizures; and gentamicin or amikacin, which are antibiotics used to treat infections, are examples of medications having a narrow margin of safety and therapeutic effectiveness with administration.

Currently available tests for therapeutic drug monitoring are invasive, difficult to administer, and/or require an extended period of time for analysis. Such tests are generally complex, requiring a laboratory to perform the analysis. Healthcare providers' offices rarely possess appropriate testing technology to analyze blood samples and must therefore send the samples to an off-site laboratory or refer the patient to the laboratory to have their blood drawn, which results in an extended time period for analysis. In the process of transfer to and from a laboratory, there is a greater likelihood that samples will be lost or mishandled, or that the incorrect results are provided to the healthcare provider, which could be detrimental to the patient's health and well-being. Further, those on-site test devices that are presently available for assessing drug concentration levels in blood are expensive. Reference laboratories using sophisticated techniques such as gas chromatography-mass spectrometry typically conduct complex and expensive toxicological analyses to determine the quantity of a medication.

It has been found that the concentration of drug in the blood may not directly reflect the concentrations at the cellular level, where most drugs exert their biological effects. The pharmacodynamics of a drug also exhibit wide inter- and intra-individual variation. The drug concentration at the site of action probably relates best with clinical responses; however, it is typically difficult or impossible to measure. Although plasma drug concentrations often provide an informative and feasible measurement for defining the pharmacodynamics of medications, they do not consistently provide an accurate report of drug disposition in a patient.

There are generally four processes by which drug disposition takes place: absorption, distribution, metabolism, and excretion. Absorption of a drug is generally dictated by route of drug administration (i.e., intravenous (IV), intramuscular (IM), subcutaneous (SC), topical, inhalation, oral, rectal, sublingual, etc.); drug factors (i.e., lipid solubility); as well as host factors (i.e., gastric emptying time). Alterations in drug absorption may affect the therapeutic effectiveness of the drug.

Factors related to drug distribution include body fat, protein binding, and membranes. Because lipid soluble drugs tend to dissolve in fat, drugs can build up to very high, potentially toxic, levels in a patient with a high percentage of body fat. There are several drugs available that have a high affinity for serum proteins. Protein binding limits the therapeutic effectiveness of the drug. Membranes such as the blood brain barrier sometimes make it difficult for the drug to be properly distributed.

All tissues in the body can contribute to the metabolism of a drug. For example, the liver, kidney, lungs, skin, brain, and gut can all be involved in metabolizing a drug. Physiologically, metabolism can increase the activity, decrease the activity, or have no effect on the activity of a drug. Because metabolism of a drug differs from one patient to another, the dosage required for a drug can differ from patient to patient.

Routes of drug elimination include the kidney, liver, gastrointestinal tract, lungs, sweat, lacrimal fluid, and milk. All of these processes (absorption, distribution, metabolism, and excretion), which can occur at varying times after drug administration, affect the level of pharmacologically effective drug in a patient. Thus, current methods for analyzing a blood sample to assess plasma drug concentrations only provides a snapshot for defining the pharmacodynamics of a drug and does not consistently provide an accurate report of drug disposition in a patient.

Accordingly, there is a need in the art for a method to improve therapeutic drug monitoring that is non-invasive, speedy, and inexpensive in administration. There is also a need for a drug monitoring system capable of continuously monitoring drug concentration levels (to assess drug disposition) as well as being used at remote locations and/or non-laboratory settings to monitor the therapeutic efficacy of the drug.

### SUMMARY OF THE INVENTION

The subject invention provides systems and methods for non-invasive monitoring of therapeutic drug concentration in blood, and, more particularly, to a system and method for the detection, quantification, and trending of delivered therapeutic drug concentration utilizing sensors that can analyze a patient's exhaled breath components.

The systems of the subject invention include at least one supply of at least one therapeutic drug for delivery to a patient; and an expired gas sensor for analyzing the patient's breath for concentration of at least one drug or marker indicative of therapeutic drugs in the patient's bloodstream, wherein the sensor provides a signal to indicate marker concentration that corresponds to therapeutic drug concentration in the patient's bloodstream.

The methods of the subject invention include the steps of measuring the concentration of one or more therapeutic markers in a patient's exhaled breath. These measured markers can then be used to quantify the concentration of therapeutic drug(s) in the patient's blood as well as trend the delivered drug, and ultimately determine the pharmacodynamics/pharmacokinetics of the drug.

In one embodiment, the subject invention contemplates administering to a patient a therapeutic drug, wherein the therapeutic drug contains a therapeutic drug marker that is detectable in exhaled breath by a sensor of the subject invention. In certain embodiments of the invention, the therapeutic drug marker is the therapeutic drug itself, which is detectable in exhaled breath. As contemplated herein, the blood concentration of the therapeutic drug and the exhaled concentration of the therapeutic drug marker are substantially proportional. By using a sensor of the subject invention for analyzing the concentration of a therapeutic drug marker in exhaled breath, which substantially corresponds to the blood concentration of a therapeutic drug, the present invention enables non-invasive, continuous monitoring of therapeutic drug blood concentration.

In a preferred embodiment of the subject invention, a specific phase of the respiratory cycle, namely the end-tidal portion of exhaled breath, is sampled to detect the concentration of a therapeutic drug marker as a measure of drug concentration levels in blood.

In accordance with the subject invention, a sensor can be selected from a variety of systems that have been developed for use in collecting and monitoring exhaled breath components, particularly specific gases. For example, the sensor of the subject invention can be selected from those described in U.S. Pat. Nos. 6,010,459; 5,081,871; 5,042,501; 4,202,352; 5,971,937, and 4,734,777. Further, sensor systems having computerized data analysis components can also be used in the subject invention (i.e., U.S. Pat. No. 4,796,639).

Sensors of the subject invention can also include commercial devices commonly known as “artificial” or “electronic” noses or tongues to non-invasively monitor therapeutic drug blood concentration. Sensors of the subject invention can include, but are not limited to, metal-insulator-metal ensemble (MIME) sensors, cross-reactive optical microsensor arrays, fluorescent polymer films, surface enhanced raman spectroscopy (SERS), semiconductor gas sensor technology, conductive polymer gas sensor technology, surface acoustic wave gas sensor technology, and immunoassays.

In certain embodiments, the systems of the subject invention include a reporting system capable of tracking marker concentration (remote or proximate) and providing the necessary outputs, controls, and alerts.

In one example, a sensor of the subject invention would be used either in a clinical setting or patient-based location during delivery of a therapeutic drug to monitor drug concentration in blood by measuring therapeutic drug marker concentration in patient exhaled breath. Moreover, exhaled breath detection using the systems and methods of the present invention may enable accurate evaluation of pharmacodynamics and pharmacokinetics for drug studies and/or in individual patients.

Therefore, it is an object of the present invention to non-invasively monitor therapeutic drug blood concentration by monitoring therapeutic drug marker concentrations in exhaled breath using sensors that analyze markers in exhaled breath. A resulting advantage of the subject invention is the ability to monitor such concentration in a more cost effective and frequent manner than current methods, which involve drawing blood samples and transferring the blood samples to a laboratory facility for analysis. In addition, the subject invention enables the user to immediately monitor therapeutic drug concentration levels in a patient's blood stream, whether in a clinical setting or via known forms of communication if the patient is located at a remote location. The systems and methods of the subject invention can be used in place of the invasive practice of drawing blood to measure concentration.

The invention will now be described, by way of example and not by way of limitation, with reference to the accompanying sheets of drawings and other objects, features and advantages of the invention will be apparent from the following detailed disclosure and from the appended claims.

### BRIEF DESCRIPTION OF THE DRAWINGS

FIG. 1 shows a capnogram of a single respiratory cycle and a capnogram of several breaths from a patient with obstructive lung disease.

FIG. 2 shows a gas sensor chip, which may be utilized as the sensor for the present invention.

### DETAILED DESCRIPTION OF THE INVENTION

The present invention provides systems and methods for non-invasive monitoring of therapeutic drug concentration in blood by analyzing therapeutic drug markers detectable in a patient's exhaled breath after administration of the therapeutic drug to the patient. Accordingly, the subject invention enables a user to provide a patient the maximum benefit from a therapeutic drug while minimizing risks for toxicity.

Definitions

As used herein, the term “therapeutic drug” or “drug” refers to a substance used in the diagnosis, treatment, or prevention of a disease or condition, wherein the concentration of the therapeutic drug in a patient's blood stream must be monitored to ensure the therapeutic drug level is within a clinically effective range.

Throughout this disclosure, a “marker” or “therapeutic drug marker” is defined as a substance that is detected by means of its physical or chemical properties using a sensor of the subject invention. According to the subject invention, therapeutic drug markers are derived either directly from the therapeutic drug itself, or from an additive combined with the therapeutic drug prior to administration. Such markers preferably include olfactory markers (odors) as well as other substances and compounds, which may be detectable by sensors of the subject invention.

A “patient,” as used herein, describes an organism, including mammals, from which exhaled breath samples are collected in accordance with the present invention. Mammalian species that benefit from the disclosed systems and methods for therapeutic drug monitoring include, and are not limited to, apes, chimpanzees, orangutans, humans, monkeys; and domesticated animals (e.g., pets) such as dogs, cats, mice, rats, guinea pigs, and hamsters.

The term “pharmacodynamics,” as used herein, refers to the interaction (biochemical and physiological) of a therapeutic drug with constituents of a patient body as well as the mechanisms of drug action on the patient body (i.e., drug effect on body).

As used herein, the term “pharmacokinetics” refers to the mathematical characterization of interactions between normal physiological processes and a therapeutic drug over time (i.e., body effect on drug). Certain physiological processes (absorption, distribution, metabolism, and elimination) will affect the ability of a drug to provide a desired therapeutic effect in a patient. Knowledge of a drug's pharmacokinetics aids in interpreting drug blood stream concentration and is useful in determining pharmacologically effective drug dosages.

“Concurrent” administration, as used herein, refers to the administration of a therapeutic drug marker suitable for use with the systems and methods of the invention (administration of a therapeutic drug) for monitoring therapeutic drug levels in blood stream. By way of example, a therapeutic drug marker can be provided in admixture with a therapeutic drug, such as in a pharmaceutical composition; or the marker and therapeutic drug can be administered to a patient as separate compounds, such as, for example, separate pharmaceutical compositions administered consecutively, simultaneously, or at different times. Preferably, if the marker and the therapeutic drug are administered separately, they are administered within sufficient time from each other so that the concentration of the marker in exhaled breath is an accurate indicator of the concentration of therapeutic drug in the blood stream.

The term “aptamer,” as used herein, refers to a non-naturally occurring oligonucleotide chain that has a specific action on a therapeutic drug marker. Aptamers include nucleic acids that are identified from a candidate mixture of nucleic acids. In a preferred embodiment, aptamers include nucleic acid sequences that are substantially homologous to the nucleic acid ligands isolated by the SELEX method. Substantially homologous is meant a degree of primary sequence homology in excess of 70%, most preferably in excess of 80%.

The “SELEX™” methodology, as used herein, involves the combination of selected nucleic acid ligands, which interact with a target marker in a desired action, for example binding to an olfactory marker, with amplification of those selected nucleic acids. Optional iterative cycling of the selection/amplification steps allows selection of one or a small number of nucleic acids, which interact most strongly with the target marker from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

As used herein, the term “pharmaceutically acceptable carrier” means a carrier that is useful in preparing a pharmaceutical composition that is generally compatible with the other ingredients of the composition, not deleterious to the patient, and neither biologically nor otherwise undesirable, and includes a carrier that is acceptable for veterinary use as well as human pharmaceutical use. “A pharmaceutically acceptable carrier” as used in the specification and claims includes both one and more than one such carrier.

Pharmacodynamics and Pharmacokinetics of Therapeutic Drugs

When a therapeutic drug is administered to a patient in accordance with the subject invention, there are many factors which effect drug pharmacodynamics and pharmacokinetics. For example, drug affinity (i.e., degree of attraction between a drug and a target receptor in the patient body), drug distribution (i.e., binding of drug to proteins circulating in the blood, absorption of drug into fat), drug metabolism and elimination (i.e., renal clearance), or existence of a drug in a “free” form may affect drug pharmacodynamics and pharmacokinetics in a patient.

A drug bound to protein or absorbed into fat does not produce a desired pharmacological effect and exists in equilibrium with unbound drug. Numerous factors, including competition for binding sites on the protein from other drugs, the amount of fat in the body, and the amount of protein produced, determine the equilibrium between bound and unbound drug.

An unbound drug can participate directly in the pharmacological effect or be metabolized into a drug that produces a desired effect. Metabolism of the active drug often leads to its removal from the bloodstream and termination of its effect. The drug effect can also be terminated by the excretion of the free drug. Free drug or a metabolite can be excreted in the urine or the digestive tract or in exhaled breath. The concentration in the blood (or plasma or serum) of such therapeutic drugs is related to the clinical effect of the agent.

As described above, blood concentration testing for a therapeutic drug may or may not provide an accurate indication of the effect of the therapeutic drug on a patient, since measurement of blood concentration does not account for the quantity of drug bound to protein or membranes, or the interaction and competition between drugs. For this reason, it would be advantageous to measure only the free drug in the plasma. The concentration of free drug in plasma is usually low and requires sophisticated and expensive analytical techniques for measurement. By contrast, the marker that appears in breath, in accordance with the subject invention, is an indication of the concentration of free drug in blood. Thus, using the systems and methods of the subject invention to measure exhaled breath for marker concentration can provide an effective indicator of the actual concentration of free drug responsible for pharmacokinetic effect.

Further, testing blood directly (i.e., drawing blood for sample analysis) is invasive, time consuming, expensive, and prone to inaccuracies. In contrast, by analyzing therapeutic drug markers in patient exhaled breath, the systems and methods of the subject invention are non-invasive, speedy, and accurate. When a therapeutic drug marker is excreted in the breath, the concentration in expired breath is proportional to the free therapeutic drug concentration in the blood and, thus, indicative of the rate of drug absorption, distribution, metabolism, and/or elimination.

In certain embodiments, a metabolite may act as a therapeutic drug marker to be measured in exhaled breath where the metabolite is a product of the active drug. As long as there is equilibrium between the active drug and a metabolite excreted in the breath, the activity of the active drug can be analyzed in accordance with the subject invention.

The method of the present invention takes into account such proportional concentrations and allows for the determination of the rate of absorption, distribution, metabolism, and elimination of a therapeutic drug by measuring concentration of unbound substances, markers, and/or active metabolites associated with the drug in a patient's breath. The proper dosing regimen can thus be determined therefrom.

Breath Sampling

Generally, the exhalation gas stream comprises sequences or stages. At the beginning of exhalation there is an initial stage, the gas representative thereof coming from an anatomically inactive (deadspace) part of the respiratory system, in other words, from the mouth and upper respiratory tracts. This is followed by a plateau stage. Early in the plateau stage, the gas is a mixture of deadspace and metabolically active gases. The last portion of the exhaled breath comprises nothing but deep lung gas, so-called alveolar gas. This gas, which comes from the alveoli, is termed end-tidal gas.

In a preferred embodiment, the exhaled breath sample is collected at end-tidal breathing. Technology similar to that used for end-tidal carbon dioxide monitoring can be used to determine when the sample is collected. Known methods for airway pressure measurements afford another means of collecting samples at the appropriate phase of the respiratory cycle. Single or multiple samples collected by the known side stream method are preferable, but if sensor acquisition time is reduced, in-line sampling may be used. In the former, samples are collected through an adapter at the proximal end of an endotracheal (ET) tube and drawn through thin bore tubing to a sensor of the subject invention.

Depending on the sample size and sensor response time, exhaled gas may be collected on successive cycles. With in-line sampling, a sensor of the subject invention is placed proximal to the ET tube directly in the gas stream. Alternatively to sample end-tidal gas, samples can be taken throughout the exhalation phase of respiration and an average value determined and correlated with blood concentration.

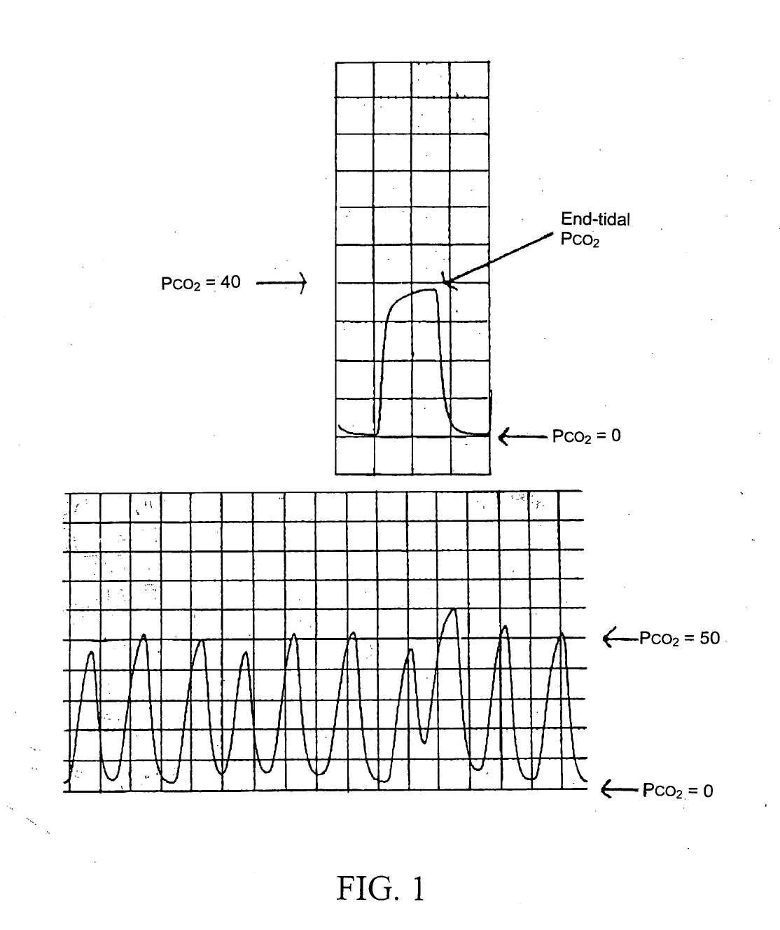

Referring now to FIG. 1, the upper frame demonstrates a capnogram of a single respiratory cycle. For accurate blood level correlation, samples are taken at the point labeled “end-tidal PCO₂” which reflects the CO₂ concentration in the lung. The lower frame shows a capnogram of several breaths from a patient with obstructive lung disease. Again the end-tidal sample correlated best with blood concentration.

In one embodiment, a VaporLab™ brand instrument is used to collect and analyze exhaled breath samples. The VaporLab™ instrument is a hand-held, battery powered SAW-based chemical vapor identification instrument suitable for detecting components in exhaled breath samples in accordance with the present invention. This instrument is sensitive to volatile and semi-volatile compounds using a high-stability SAW sensor array that provides orthogonal vapor responses for greater accuracy and discrimination. In a related embodiment, this instrument communicates with computers to provide enhanced pattern analysis and report generation. In a preferred embodiment, this instrument includes neural networks for “training” purposes, i.e., to remember chemical vapor signature patterns for fast, “on-the-fly” analysis.

In another embodiment, samples are collected at the distal end of an ET tube through a tube with a separate sampling port. This may improve sampling by allowing a larger sample during each respiratory cycle.

In certain instances, the concentration of a therapeutic drug in a patient body is regulated by the amount of the drug administered over a given time period and the rate at which the agent is eliminated from the body (metabolism). The present invention provides the steps of administering a therapeutic drug to a patient and analyzing patient exhaled breath for concentration of therapeutic drug markers such as unbound substances, active metabolites, or inactive metabolites associated with the therapeutic drug, after a suitable time period. In certain embodiments of the subject invention, the marker concentration indicates a characteristic of metabolism of the drug in the patient.

Methods of the subject invention may further include the use of a flow sensor to detect starting and completion of exhalation. The method further includes providing results from the analysis and communicating to the user or patient the blood concentration of the therapeutic drug. In a preferred embodiment, results from analysis can be communicated immediately upon sampling exhaled gases.

In certain embodiments, the subject invention enables the immediate monitoring of therapeutic drug levels in a patient's blood stream. As contemplated herein, immediate monitoring refers to sampling and analysis of exhaled gases from a patient for target markers substantially completely within a short time period following administration of a therapeutic drug (i.e., generally within a few minutes to about 24 hours).

Alternatively, in certain instances, a specific period of time must progress before a therapeutic drug concentration level in the blood stream can be detected. Accordingly, a system and/or method of the invention can be provided to a patient taking a therapeutic drug for intermittent or continuous monitoring of therapeutic drug concentrations in the blood stream. In certain embodiments, the monitoring system and method of the subject invention can be administered to a patient taking a therapeutic drug on an hourly, daily, weekly, monthly, or even annual basis. Further, additional monitoring can be administered to a patient when an additional therapeutic drug is prescribed.

Moreover, a CPU may be provided as a data processing/control unit for automatically detecting the signal from the flow sensor to control sampling of exhaled breath. The CPU may further provide to the user/patient the appropriate dosage of the therapeutic drug to be delivered based on analysis of trends in therapeutic drug blood concentration.

Depending on the mode of therapeutic drug administration, the present invention provides means for automatically adjusting and administering the appropriate dosage of a therapeutic drug, based on blood concentration levels, to a patient. In certain embodiments, a CPU is provided for analysis and control of dosage adjusting and administering means. In one embodiment in which a therapeutic drug is delivered intravenously, an infusion pump is used, wherein the CPU provides analysis and control of the infusion pump.

Concentration in the blood of therapeutic drug markers, as measured by breath analysis in accordance with the present invention, may indicate when the patient is receiving a high dose (i.e., toxic dose), a low dose (i.e., ineffective dose), or effective (i.e., appropriate) dose of the therapeutic drug. Even if there is wide variation in the metabolism or response to the therapeutic drug, knowledge of the exhaled breath concentration allows the user to know if the drug is accumulating in the blood, possibly leading to dangerously toxic levels of the drug, or that the concentration is falling, possibly leading to an inadequate dose of the drug. Monitoring changes in therapeutic drug blood concentration in accordance with the subject invention are, therefore, useful.

In another embodiment, the exhalation air is measured for marker concentration either continuously or periodically. From the exhalation air is extracted at least one measured marker concentration value. Numerous types of breath sampling apparatuses can be used to carry out the method of the present invention.

In one embodiment, the breath sampling apparatus includes a conventional flow channel through which exhalation air flows. The flow channel is provided with a sensor of the subject invention for measuring marker concentration. Furthermore, necessary output elements may be included with the breath sampling apparatus for delivering at least a measured concentration result to the user, if necessary.

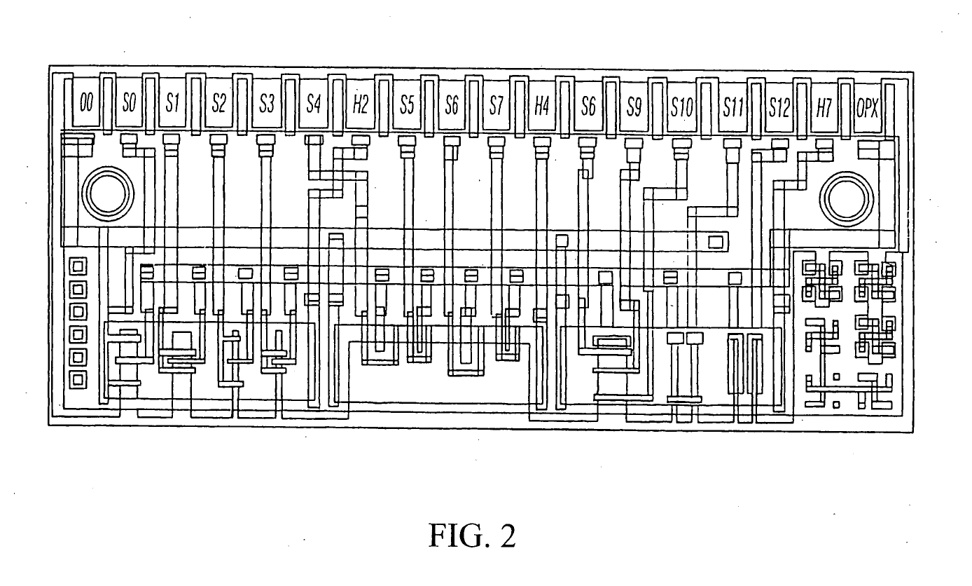

An alarm mechanism may also be provided. An instrument of similar type is shown in FIGS. 1 and 2 of U.S. Pat. No. 5,971,937 incorporated herein by reference.

In another embodiment, once the level of concentration is measured, it is given numerical value (for example, 50 on a scale of 1 to 100). Should the concentration fall below that value, the new value would be indicative of a decrease in concentration. Should the concentration increase beyond that value, the new value would be indicative of an increase in concentration. This numerical scale would allow for easier monitoring of changes in concentration. The numerical scale would also allow for easier translation into control signals for alarms, outputs, charting, and control of external devices (e.g., infusion pump). The upper and lower limits could be set to indicate thresholds such as from ineffective to dangerous therapeutic drug levels.

Sensor Technology

The invention preferably utilizes gas sensor technology, such as commercial devices known as “artificial” or “electronic” tongues or noses, to non-invasively monitor marker concentration in exhaled breath (FIG. 2). Electronic noses have been used mostly in the food, wine, and perfume industry where their sensitivity makes it possible to distinguish between odorous compounds. For example, electronic noses have been useful in distinguishing between grapefruit oil and orange oil in the perfume industry and identify spoilage in perishable foods before the odor is evident to the human nose.

In the past, there was little medical-based research and application of these artificial/electronic tongues and noses. However, recent use has demonstrated the power of this non-invasive technique. For example, electronic noses have been used to determine the presence of bacterial infection in the lungs by analyzing the exhaled gases of patients for odors specific to particular bacteria (Hanson C W, Steinberger H A, “The use of a novel electronic nose to diagnose the presence of intrapulmonary infection,” Anesthesiology, 87(3A): Abstract A269, (1997)). Also, a genitourinary clinic has utilized an electronic nose to screen for, and detect bacterial vaginosis, with a 94% success rate after training (Chandiok S, et al., “Screening for bacterial vaginosis: a novel application of artificial nose technology,” Journal of Clinical Pathology, 50(9):790-1 (1997)). Specific bacterial species can also be identified with the electronic nose based on special odors produced by the organisms (Parry AD et al., “Leg ulcer odor detection identifies beta-haemolytic streptococcal infection,” Journal of Wound Care, 4:404-406 (1995)).

A number of patents which describe gas sensor technology that can be used in the subject invention include, but are not limited to, the following: U.S. Pat. Nos. 5,945,069; 5,918,257; 4,938,928; 4,992,244; 5,034,192; 5,071,770; 5,145,645; 5,252,292; 5,605,612; 5,756,879; 5,783,154; and 5,830,412. Other sensors suitable for the present invention include, but are not limited to, metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave sensors, calorimetric tubes, infrared spectroscopy.

Recent developments in the field of detection that can also be used as sensor for the subject invention include, but are not limited to, gas chromatography, semiconductive gas sensors, mass spectrometers (including proton transfer reaction mass spectrometry), and infrared (IR) or ultraviolet (UV) or visible or fluorescence spectrophotometers (i.e., non-dispersive infrared spectrometer). For example, with semiconductive gas sensors, markers cause a change in the electrical properties of semiconductor(s) by making their electrical resistance vary, and the measurement of these variations allows one to determine the concentration of marker(s). In another example, gas chromatography, which consists of a method of selective detection by separating the molecules of gas compositions, may be used as a means for analyzing markers in exhaled breath samples.

In accordance with the subject invention, sensors for detecting/quantifying markers utilize a relatively brief detection time of around a few seconds. Other recent gas sensor technologies contemplated by the present invention include apparatuses having conductive-polymer gas-sensors (“polymeric”), aptamer biosensors, amplifying fluorescent polymer (AFP) sensors, and apparatuses having surface-acoustic-wave (SAW) gas-sensors.

The conductive-polymer gas-sensors (also referred to as “chemoresistors”) have a film made of a conductive polymer sensitive to the molecules of odorous substances. On contact with target marker molecules, the electric resistance of the sensors changes and the measurement of the variation of this resistance enables the concentration of the markers to be determined. An advantage of this type of sensor is that it functions at temperatures close to room temperature. Different sensitivities for detecting different markers can be obtained by modifying or choosing an alternate conductive polymer.

Polymeric gas sensors can be built into an array of sensors, where each sensor is designed to respond differently to different markers and augment the selectivity of the therapeutic drug markers. For example, a sensor of the subject invention can comprise of an array of polymers, (i.e., 32 different polymers) each exposed to a marker. Each of the individual polymers swells differently to the presence of a marker, creating a change in the resistance of that membrane and generating an analog voltage in response to that specific marker (“signature”). The normalized change in resistance can then be transmitted to a processor to identify the type, quantity, and quality of the marker based on the pattern change in the sensor array. The unique response results in a distinct electrical fingerprint that is used to characterize the marker. The pattern of resistance changes of the array is diagnostic of the marker in the sample, while the amplitude of the pattern indicates the concentration of the marker in the sample.

Another sensor of the invention can be provided in the form of an aptamer. In one embodiment, the SELEX™ (Systematic Evolution of Ligands by EXponential enrichment) methodology is used to produce aptamers that recognize therapeutic drug markers with high affinity and specificity. Aptamers produced by the SELEX methodology have a unique sequence and the property of binding specifically to a desired marker. The SELEX methodology is based on the insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. According to the subject invention, therapeutic drug markers of any size or composition can thus serve as targets for aptamers. See also Jayasena, S., “Aptamers: An Emerging Class of Molecules That Rival Antibodies for Diagnostics,” Clinical Chemistry, 45:9, 1628-1650 (1999).

Aptamer biosensors can be utilized in the present invention for detecting the presence of markers in exhaled breath samples. In one embodiment, aptamer sensors are composed of resonant oscillating quartz sensors that can detect minute changes in resonance frequencies due to modulations of mass of the oscillating system, which results from a binding or dissociation event (i.e., binding with a target therapeutic drug marker).

Similarly, amplifying fluorescent polymer (AFP) sensors may be utilized in the present invention for detecting the presence of therapeutic drug markers in exhaled breath samples. AFP sensors are extremely sensitive and highly selective chemosensors that use amplifying fluorescent polymers. When vapors bind to thin films of the polymers, the fluorescence of the film decreases. A single molecule binding event quenches the fluorescence of many polymer repeat units, resulting in an amplification of the quenching. The binding of markers to the film is reversible, therefore the films can be reused.

Surface-acoustic-wave (SAW) sensors oscillate at high frequencies and generally have a substrate, which is covered by a chemoselective material. In SAW sensors, the substrate is used to propagate a surface acoustic wave between sets of interdigitated electrodes (i.e., to form a transducer). The chemoselective material is coated on the transducer. When a marker interacts with the chemoselective material coated on the substrate, the interaction results in a change in the SAW properties, such as the amplitude of velocity of the propagated wave. The detectable change in the characteristic wave is generally proportional to the mass load of the marker(s) (i.e., concentration of the marker in exhaled breath, which corresponds to the concentration of the therapeutic drug in the blood stream).

Certain embodiments of the invention use known SAW devices, such as those described in U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator adsorbent,” Analytica Chimica Acta, 371:131-143 (1988). Other types of chemical sensors known in the art that use chemoselective coating applicable to the operation of the present invention include bulk acoustic wave (BAW) devices, plate acoustic wave devices, interdigitated microelectrode (IME) devices, optical waveguide (OW) devices, electrochemical sensors, and electrically conducting sensors.

In one embodiment, the sensor of the invention is based on surface acoustic wave (SAW) sensors. The SAW sensors preferably include a substrate with piezoelectric characteristics covered by a polymer coating, which is able to selectively absorb target markers. SAW sensors oscillate at high frequencies and respond to perturbations proportional to the mass load of certain molecules. This occurs in the vapor phase on the sensor surface.

In a related embodiment, the sensor of the invention is based on a SAW sensor of Stubbs, D. et al. (see Stubbs, D. et al., “Investigation of cocaine plumes using surface acoustic wave immunoassay sensors,” Anal Chem., 75(22):6231-5 (November 2003) and Stubbs, D. et al., “Gas phase activity of anti-FITC antibodies immobilized on a surface acoustic wave resonator device,” Biosens Bioelectron, 17(6-7):471-7 (2002)). For example, the sensor of the subject invention can include a two-port resonator on ST-X quartz with a center frequency of 250 MHz. On the cut quartz, a temperature compensated surface acoustic wave (SAW) is generated via an interdigital transducer. Antibodies specific to a target marker are then attached to the electrodes (i.e., 1.5 micron wide) on the sensor device surface via protein cross linkers. In the vapor phase on the sensor surface, when target markers are present, a change in frequency occurs to alert the user that a target marker has been recognized.

In a related embodiment, the SAW sensor is connected to a computer, wherein any detectable change in frequency can be detected and measured by the computer. In a preferred embodiment, an array of SAW sensors (4-6) is used, each coated with a different chemoselective polymer that selectively binds and/or absorbs vapors of specific classes of molecules. The resulting array, or “signature” identifies specific compounds.

The operating performance of most chemical sensors that use a chemoselective film coating is greatly affected by the thickness, uniformity and composition of the coating. For these sensors, increasing the coating thickness, has a detrimental effect on the sensitivity. Only the transducer senses the portion of the coating immediately adjacent to the transducer/substrate.

For example, if the polymer coating is too thick, the sensitivity of a SAW device to record changes in frequency will be reduced. These outer layers of coating material compete for the marker with the layers of coating being sensed and thus reduce the sensitivity of the sensor. Uniformity of the coating is also a critical factor in the performance of a sensor that uses a chemoselective coating since changes in average surface area greatly affect the local vibrational signature of the SAW device. Therefore, films should be deposited that are flat to within 1 nm with a thickness of 15-25 nm. In this regard, it is important not only that the coating be uniform and reproducible from one device to another, so that a set of devices will all operate with the same sensitivity, but also that the coating on a single device be uniform across the active area of the substrate.

If a coating is non-uniform, the response time to marker exposure and the recovery time after marker exposure are increased and the operating performance of the sensor is impaired. The thin areas of the coating respond more rapidly to a target marker than the thick areas. As a result, the sensor response signal takes longer to reach an equilibrium value, and the results are less accurate than they would be with a uniform coating.

Most current technologies for creating large area films of polymers and biomaterials involve the spinning, spraying, or dipping of a substrate into a solution of the macromolecule and a volatile solvent. These methods coat the entire substrate without selectivity and sometimes lead to solvent contamination and morphological inhomogeneities in the film due to non-uniform solvent evaporation. There are also techniques such as microcontact printing and hydrogel stamping that enable small areas of biomolecular and polymer monolayers to be patterned, but separate techniques like photolithography or chemical vapor deposition are needed to transform these films into microdevices.

Other techniques such as thermal evaporation and pulsed laser ablation are limited to polymers that are stable and not denatured by vigorous thermal processes. More precise and accurate control over the thickness and uniformity of a film coating may be achieved by using pulsed laser deposition (PLD), a physical vapor deposition technique that has been developed recently for forming ceramic coatings on substrates. By this method, a target comprising the stoichiometric chemical composition of the material to be used for the coating is ablated by means of a pulsed laser, forming a plume of ablated material that becomes deposited on the substrate.

Polymer thin films, using a new laser based technique developed by researchers at the Naval Research Laboratory called Matrix Assisted Pulsed Laser Evaporation (MAPLE), have recently been shown to increase sensitivity and specificity of chemoselective Surface Acoustic Wave vapor sensors. A variation of this technique, Pulsed Laser Assisted Surface Functionalization (PLASF) is preferably used to design compound specific biosensor coatings with increased sensitivity for the present invention. PLASF produces similar thin films for sensor applications with bound receptors for biosensor applications. By providing improved SAW biosensor response by eliminating film imperfections induced by solvent evaporation and detecting molecular attachments to specific target markers, high sensitivity and specificity is possible.

Certain extremely sensitive, commercial off-the-shelf (COTS) electronic noses, such as those provided by Cyrano Sciences, Inc. (“CSI”) (i.e., CSI's Portable Electronic Nose and CSI's Nose-Chip integrated circuit for odor-sensing, see U.S. Pat. No. 5,945,069— FIG. 1), may be used in the system and method of the present invention to monitor the exhaled breath from a patient. These devices offer minimal cycle time, can detect multiple markers, can work in almost any environment without special sample preparation or isolation conditions, and do not require advanced sensor design or cleansing between tests.

In one embodiment, the device of the present invention may be designed so that patients can exhale via the mouth or nose directly onto a sensor of the invention. In another embodiment, a patient's breath sample can be captured in a container (vessel) for later analysis using a sensor of the subject invention (i.e., mass spectrometer).

The results from the sensor technology analysis of the bodily fluid samples are optionally provided to the user (or patient) via a reporting means. In one embodiment, the sensor technology includes the reporting means. Contemplated reporting means include a computer processor linked to the sensor technology in which electronic or printed results can be provided. Alternatively, the reporting means can include a digital display panel, transportable read/write magnetic media such as computer disks and tapes_which can be transported to and read on another machine, and printers such as thermal, laser or ink-jet printers for the production of a printed report.

The reporting means can provide the results to the user (or patient) via facsimile, electronic mail, mail or courier service, or any other means of safely and securely sending the report to the patient. Interactive reporting means are also contemplated by the present invention, such as an interactive voice response system, interactive computer-based reporting system, interactive telephone touch-tone system, or other similar system. The report provided to the user (or patient) may take many forms, including a summary of analyses performed over a particular period of time or detailed information regarding a particular bodily fluid sample analysis. Results may also be used to populate a financial database for billing the patient, or for populating a laboratory database or a statistical database.

A data monitor/analyzer can compare a pattern of response to previously measured and characterized responses from known markers. The matching of those patterns can be performed using a number of techniques, including neural networks. By comparing the analog output from each of the 32 polymers to a “blank” or control, for example, a neural network can establish a pattern that is unique to that marker and subsequently learns to recognize that marker. The particular resistor geometries are selected to optimize the desired response to the target marker being sensed. The sensor of the subject invention is preferably a self-calibrating polymer system suitable for detecting and quantifying markers in gas phase biological solutions to assess and/or monitor a variety of therapeutic drug markers simultaneously.

According to the subject invention, the sensor can include a computer that communicates therewith, which can also notify the medical staff and/or the patient as to any irregularities in dosing, dangerous drug interactions, and the like. This system will enable determination as to whether a patient has been administered a pharmacologically effective amount of a therapeutic drug. The device could also alert the patient (or user) as to time intervals and/or dosage of therapeutic drug to be administered. Accordingly, it is contemplated herein that a sensor of the subject invention can be portable.

The sensor of the present invention might include integrated circuits (chips) manufactured in a modified vacuum chamber for Pulsed Laser Deposition of polymer coatings. It will operate the simultaneous thin-film deposition wave detection and obtain optimum conditions for high sensitivity of SAW sensors. The morphology and microstructure of biosensor coatings will be characterized as a function of process parameters.

The sensor used in the subject invention may be modified so that patients can exhale directly onto the sensor, without needing a breath sampling apparatus. For example, a mouthpiece or nosepiece will be provided for interfacing a patient with the device to readily transmit the exhaled breath to the sensor (See, i.e., U.S. Pat. No. 5,042,501). In a related embodiment, wherein the sensor is connected to a neural network, the output from the neural network is similar when the same patient exhales directly into the device and when the exhaled gases are allowed to dry before the sensor samples them.

The humidity in the exhaled gases represents a problem for certain electronic nose devices (albeit not SAW sensors) that only work with “dry” gases. When using such humidity sensitive devices, the present invention may adapt such electronic nose technology so that a patient can exhale directly into the device with a means to dehumidify the samples. This is accomplished by including a commercial dehumidifier or a heat moisture exchanger (HME), a device designed to prevent desiccation of the airway during ventilation with dry gases.

Alternatively, the patient may exhale through their nose, which is an anatomical, physiological dehumidifier to prevent dehydration during normal respiration. Alternatively, the sensor device can be fitted with a preconcentrator, which has some of the properties of a GC column. The gas sample is routed through the preconcentrator before being passed over the sensor array. By heating and volatilizing the gases, humidity is removed and the marker being measured can be separated from potential interferents.

Preferably, in operation, the sensor will be used to identify a baseline spectrum for the patient prior to drug administration, if necessary. This will prove beneficial for the detection of more than one therapeutic drug if the patient receives more than one drug at a time and possible interference from different foods and odors in the stomach, mouth, esophagus and lungs.

Therapeutic Drug Markers

In accordance with the present invention, therapeutic drug markers useful as an indication of therapeutic drug concentration in blood include the following olfactory markers, without limitation: dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene). These markers are preferred since they are used in the food industry as flavor ingredients and are permitted by the Food and Drug Administration. As indicated above, olfactory markers for use in the present invention can be selected from a vast number of available compounds (see Fenaroli's Handbook of Flavor Ingredients, 4th edition, CRC Press, 2001) and use of such other applicable markers is contemplated herein.

The markers of the invention also include additives that have been federally approved and categorized as GRAS (“generally recognized as safe”), which are available on a database maintained by the U.S. Food and Drug Administration Center for Food Safety and Applied Nutrition. Markers categorized as GRAS that are readily detectable in exhaled breath include, but are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

As described above, therapeutic drug markers are detected by their physical and/or chemical properties, which does not preclude using the desired therapeutic drug itself as its own marker. Therapeutic drug markers, as contemplated herein, also include products and compounds that are administered to enhance detection using sensors of the invention. Moreover, therapeutic drug markers can include a variety of products or compounds that are added to a desired therapeutic drug regimen to enhance differentiation in detection/quantification. Generally, in accordance with the present invention, therapeutic drug markers are poorly soluble in water, which enhances their volatility and detection in the breath.

According to the subject invention, upon administering a therapeutic drug (wherein the therapeutic drug is the marker) or upon concurrent administration of a therapeutic drug and marker, marker detection can occur under several circumstances. In one example where the drug is administered orally, the marker can “coat” or persist in the mouth, esophagus and/or stomach upon ingestion and be detected with exhalation (similar to the taste or flavor that remains in the mouth after eating a breath mint).

In a second instance where the drug (and marker) is administered orally, the drug may react in the mouth or stomach with acid or enzymes to produce or liberate the marker that can then be detected upon exhalation. Thirdly, the drug and/or marker can be absorbed in the gastrointestinal tract and be excreted in the lungs (i.e. alcohol is rapidly absorbed and detected with a Breathalyzer). Generally, a therapeutic drug marker of the invention provides a means for determining the pharmacodynamics and pharmacokinetics of the drug.

In one embodiment, a therapeutic drug marker is concurrently administered with a therapeutic drug (i.e., marker is provided in a pharmaceutically acceptable carrier—marker in medication coating composed of rapidly dissolving glucose and/or sucrose. In a preferred embodiment, the therapeutic drug is provided in the form of a pill, whose coating includes at least one marker in air-flocculated sugar crystals. This would stimulate salivation and serve to spread the marker around the oral cavity, enhancing the lifetime in the cavity. Since the throat and esophagus could also be coated with the marker as the medication is ingested, detection of the marker is further enhanced.

Thus, when a drug is administered to a patient, the preferred embodiment of the invention detects and quantifies a therapeutic drug marker almost immediately in the exhaled breath of the patient (or possibly by requesting the patient to deliberately produce a burp) using a sensor (i.e., electronic nose). Certain drug compositions might not be detectable in the exhaled breath. Others might have a coating to prevent the medication from dissolving in the stomach. In both instances, as an alternate embodiment, a non-toxic olfactory marker (i.e., volatile organic vapors) can be added to the pharmaceutically acceptable carrier (i.e., the coating of a pill, in a separate fast dissolving compartment in the pill, or solution, if the drug is administered in liquid or suspension form) to provide a means for identifying/quantifying the marker in exhaled breath and thus determine the drug concentration in blood.

Preferably the marker will coat the oral cavity or esophagus or stomach for a short while and be exhaled in the breath (or in a burp). For drugs administered in the form of pills, capsules, and fast-dissolving tablets, the markers can be applied as coatings or physically combined or added to therapeutic drug. Markers can also be included with therapeutic drugs that are administered in liquid form (i.e., syrups, via inhalers, or other dosing means).

The markers of the invention could be used for indicating specific drugs or for a class of drugs. For example, a patient may be taking an anti-depressant (tricyclics such as nortriptyline), antibiotic, an antihypertensive agent (i.e., clonidine), pain medication, and an anti-reflux drug. One marker could be used for antibiotics as a class, or for subclasses of antibiotics, such as erythromycins. Another marker could be used for antihypertensives as a class, or for specific subclasses of antihypertensives, such as calcium channel blockers. The same would be true for the anti-reflux drug. Furthermore, combinations of marker substances could be used allowing a rather small number of markers to specifically identify a large number of medications.

Remote Communication System

A further embodiment of the invention includes a communications device in the home (or other remote location) that will be interfaced to the sensor. The home communications device will be able to transmit immediately or at prescribed intervals directly or over a standard telephone line (or other communication transmittal means) the data collected by the data monitor/analyzer device. The communication of the data will allow the user (i.e., physician) to be able to remotely verify if the appropriate dosage of a therapeutic drug is being administered to the patient. The data transmitted from the home can also be downloaded to a computer where the drug blood levels are stored in a database, and any deviations outside of pharmacological efficacy would be automatically flagged (ie., alarm) so that a user (i.e., patient, physician, nurse) could appropriately adjust the drug dosage per suggestions provided by a computer processing unit connected to the sensor or per dosage suggestions provided by health care personnel (i.e., physician).

Therapeutic Drugs

As contemplated herein, therapeutic drugs to be monitored in accordance with the subject invention include, but are not limited to, psychiatric drugs (i.e., antidepressants, anti-psychotics, anti-anxiety drugs, depressants), analgesics, stimulants, biological response modifiers, NSAIDs, corticosteroids, DMARDs, anabolic steroids, antacids, antiarrhythmics, antibacterials, antibiotics, anticoagulants and thrombolytics, anticonvulsants, antidiarrheals, antiemetics, antihistamines, antihypertensives, anti-inflammatories, antineoplastics, antipyretics, antivirals, barbiturates, β-blockers, bronchodilators, cough suppressants, cytotoxics, decongestants, diuretics, expectorants, hormones, immunosuppressives, hypoglycemics, laxatives, muscle relaxants, sedatives, tranquilizers, and vitamins.

For example, the subject invention can effectively monitor concentrations of the following non-limiting list of therapeutic drugs in blood: drugs for the treatment of rheumatoid arthritis or symptoms thereof, systemic lupus erythematosus or symptoms thereof, degenerative arthritis, vasculitis, inflammatory diseases, angina, coronary artery disease, peripheral vascular disease; ulcerative colitis, and Crohn's disease; anti organ rejection drugs; antiepilepsy medication; and anti-anxiety drugs.

Therapeutic drugs whose concentration levels in blood can be monitored in accordance with the subject invention include, but are not limited to, the following: α-Hydroxy-Alprazolam; Acecainide (NAPA); Acetaminophen (Tylenol); Acetylmorphine; Acetylsalicylic Acid (as Salicylates); α-hydroxy-alprazolam; Alprazolam (Xanax); Amantadine (Symmetrel); Ambien (Zolpidem); Amikacin (Amikin); Amiodarone (Cordarone); Amitriptyline (Elavil) & Nortriptyline; Amobarbital (Amytal); Anafranil (Clomipramine) & Desmethylclomipramine; Ativan (Lorazepam); Aventyl (Nortriptyline); Benadryl (Dephenhydramine); Benziodiazepines; Benzoylecgonine; Benztropine (Cogentin); Bupivacaine (Marcaine); Bupropion (Wellbutrin) and Hydroxybupropion; Butabarbital (Butisol); Butalbital (Fiorinal) Carbamazepine (Tegretol); Cardizem (Diltiazem); Carisoprodol (Soma) & Meprobamate; and Celexa (Citalopram & Desmethylcitalopram).

Additional therapeutic drugs whose blood concentration levels can be monitored in accordance with the subject invention include Celontin (Methsuximide) (as desmethylmethsuximide); Centrax (Prazepam) (as Desmethyldiazepam); Chloramphenicol (Chloromycetin); Chlordiazepoxide; Chlorpromazine (Thorazine); Chlorpropamide (Diabinese); Clonazepam (Kionopin); Clorazepate (Tranxene); Clozapine; Cocaethylene; Codeine; Cogentin (Benztropine); Compazine (Prochlorperazine); Cordarone (Amiodarone); Coumadin (Warfarin); Cyclobenzaprine (Flexeril); Cyclosporine (Sandimmune); Cylert (Pemoline); Dalmane (Flurazepam) & Desalkylflurazepam; Darvocet; Darvon (Propoxyphene) & Norpropoxyphene; Demerol (Meperidine) & Normeperidine; Depakene (Valproic Acid); Depakote (Divalproex) (Measured as Valproic Acid); Desipramine (Norpramin); Desmethyldiazepam; Desyrel (Trazodone); Diazepam & Desmethyldiazepam; Diazepam (Valium) Desmethyldiazepam; Dieldrin; Digoxin (Lanoxin); Dilantin (Phenyloin); Disopyramide (Norpace); Dolophine (Methadone); Doriden (Glutethimide); Doxepin (Sinequan) and Desmethyldoxepin; Effexor (Venlafaxine); Ephedrine; Equanil (Meprobamate) Ethanol; Ethosuximide (Zarontin); Ethotoin (Peganone); Felbamate (Felbatol); Fentanyl (Innovar); Fioricet; Fipronil; Flunitrazepam (Rohypnol); Fluoxetine (Prozac) & Norfluoxetine; Fluphenazine (Prolixin); Fluvoxamine (Luvox); Gabapentin (Neurontin); Gamma-Hydroxybutyric Acid (GHB); Garamycin (Gentamicin); Gentamicin (Garamycin); Halazepam (Paxipam); Halcion (Triazolam); Haldol (Haloperidol); Hydrocodone (Hycodan); Hydroxyzine (Vistaril); Ibuprofen (Advil, Motrin, Nuprin, Rufen); Imipramine (Tofranil) and Desipramine; Inderal (Propranolol); Keppra (Levetiracetam); Ketamine; Lamotrigine (Lamictal); Lanoxin (Digoxin); Lidocaine (Xylocalne); Lindane (Gamma-BHC); Lithium; Lopressor (Metoprolol); Lorazepam (Ativan); and Ludiomil.

Blood level concentrations of the following therapeutic drugs that can be monitored in accordance with the subject invention include, but are not limited to, Maprotiline; Mebaral (Mephobarbital) & Phenobarbital; Mellaril (Thioridazine) & Mesoridazine; Mephenyloin (Mesantoin); Meprobamate (Miltown, Equanil); Mesantoin (Mephenyloin); Mesoridazine (Serentil); Methadone; Methotrexate (Mexate); Methsuximide (Celontin) (as desmethsuximide); Mexiletine (Mexitil); Midazolam (Versed); Mirtazapine (Remeron); Mogadone (Nitrazepam); Molindone (Moban); Morphine; Mysoline (Primidone) & Phenobarbital; NAPA & Procainamide (Pronestyl); NAPA (N-Acetyl-Procainamide); Navane (Thiothixene); Nebcin (Tobramycin); Nefazodone (Serzone); Nembutal (Pentobarbital); Nordiazepam; Olanzapine (Zyprexa); Opiates; Orinase (Tolbutamide); Oxazepam (Serax); Oxcarbazepine (Trileptal) as 10-Hydroxyoxcarbazepine; Oxycodone (Percodan); Oxyrnorphone (Numorphan); Pamelor (Nortriptyline); Paroxetine (Paxil); Paxil (Paroxetine); Paxipam (Halazepam); Peganone (Ethotoin); PEMA (Phenylethylmalonamide); Pentothal (Thiopental); Perphenazine (Trilafon); Phenergan (Promethazine); Phenothiazine; Phentermine; Phenylglyoxylic Acid; Procainamide (Pronestyl) & NAPA; Promazine (Sparine); Propafenone (Rythmol); Protriptyline (Vivactyl); Pseudoephedrine; Quetiapine (Seroquel); Restoril (Temazepam); Risperdal (Risperidone) and Hydroxyrisperidone; Secobarbital (Seconal); Sertraline (Zoloft) & Desmethylsertraline; Stelazine (Trifluoperazine); Surmontil (Trimipramine); Tocainide (Tonocard); and Topamax (Topiramate).

Therapeutic drugs of the subject invention can be formulated according to known methods for preparing pharmaceutically useful compositions. Formulations are described in a number of sources; which are well known and readily available to those skilled in the art. For example, Remington 's Pharmaceutical Science (Martin E W [1995] Easton Pa., Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents.

Formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Administration of a therapeutic drug, in accordance with the subject invention, can be accomplished by any suitable method and technique presently or prospectively known to those skilled in the art. In a preferred embodiment, a therapeutic drug is formulated in a patentable and easily consumed oral formulation such as a pill, lozenge, tablet, gum, beverage, etc.

According to the subject invention, a therapeutic drug can be delivered from a controlled supply means (i.e., pill dispenser, IV bag, etc.). Upon delivery of the therapeutic drug to a patient, a sensor of the invention analyzes a patient's expired gases to detect at least one target marker of the therapeutic drug. Upon detection of the target marker, the concentration of the therapeutic drug in blood can be determined for use in deriving the appropriate dosage amount of the therapeutic drug to next be delivered to the patient. In one embodiment, a system controller utilizes the derived appropriate dosage based on exhaled breath analysis to dispense an appropriate dosage from the supply means to the patient.

Additional embodiments are also envisioned herein. Pulmonary delivery of medications is well known, especially for conditions such as asthma and chronic obstructive pulmonary disease. In these instances, medication (i.e. corticosteroids, bronchodilators, anticholenergics, etc.) is often nebulized or aerosolized and inhaled through the mouth directly into the lungs. This allows delivery directly to the affected organ (the lungs) and reduces side effects common with enteral (oral) delivery. Metered dose inhalers (MDIs) or nebulizers are commonly used to deliver medication by this route. Recently dry powder inhalers have become increasingly popular, as they do not require the use of propellants such as CFCs. Propellants have been implicated in worsening asthma attacks, as well as depleting the ozone layer. Dry power inhalers are also being used for drugs that were previously given only by other routes, such as insulin, peptides, and hormones.

Olfactory markers can be added to these delivery systems as well. Since the devices are designed to deliver medication by the pulmonary route, the sensor array can be incorporated into the device and the patient need only exhale back through the device for documentation to occur.

Lastly, devices are available to deliver medication by the intranasal route. This route is often used for patients with viral infections or allergic rhinitis, but is being increasing used to deliver peptides and hormones as well. Again, it would be simple to incorporate a sensor array into these devices, or the patient can exhale through the nose for detection by a marker sensing system.

### EXAMPLE 1

### Estimation of Free Blood Propofol Concentration During Intravenous Administration by Measurement of Exhaled Breath Propofol with a SAW-Based Sensor System of the Invention

Propofol, an intravenous anesthetic agent, is frequently administered by continuous infusion to provide sedation to patients in the intensive care unit (ICU). Propofol is extremely lipophilic and also binds strongly to proteins and red blood cells. It is estimated that only 1-3% of propofol is free in plasma. It is this free fraction of propofol that is responsible for the desired therapeutic effect.

Often during a clinical procedure, it is desirable to periodically stop the propofol infusion to perform neurological examinations on patients, particularly those who have suffered a brain injury. Unfortunately, depending on the pharmacodynamics of propofol in an individual patient, the free blood concentration can be greater or less than that estimated by population pharmacodynamics and pharmacokinetics. This can lead to inadequate sedation, which may result in agitation and additional brain insult, or to accumulation of propofol in adipose tissue, resulting in prolonged sedation or even anesthesia, preventing adequate neurological examination.

The subject invention overcomes these deficiencies in the use of propofol. By continuously monitoring the end-tidal exhaled breath propofol concentration, an infusion pump can be programmed and regulated to maintain a precise exhaled breath, and thus, blood concentration of propofol. This will allow the healthcare provider to maintain the patient in a precise plane of sedation or anesthesia and overcome many of the complications related to using propofol for long periods of time where it might accumulate in adipose tissue and/or compete for binding sites on proteins and red blood cells.

### EXAMPLE 2

### Estimation of Antibiotic Blood Concentrations Using Exhaled Breath Measurements as a Surrogate

Patients requiring intravenous antibiotics for serious infections often require frequent blood sampling to obtain antibiotic concentrations. Often “peak” and “trough” levels are drawn to insure that the blood concentration of drug is adequate just prior to giving the next dose. Inadequate blood levels can predispose to bacteria developing drug resistance. A sensor for analyzing antibiotic markers in exhaled breath can be calibrated against a peak and trough level and for all subsequent measurements for use as a surrogate for measuring blood antibiotic levels and to subsequently direct therapy.

### EXAMPLES 3

### Exhaled Breath Anti-Seizure Medication Levels as a Surrogate for Blood Concentration.

Patients taking anti-seizure medications require frequent testing and analysis of blood samples to determine the concentration of the medication in their blood. Many anti-seizure medications have a narrow therapeutic range and low blood levels can lead to an increased frequency of seizures, while high levels can lead to significant toxicity. A sensor for detecting in exhaled breath anti-seizure medication markers can be calibrated against the blood anti-seizure medication concentration and used to monitor blood levels without the patient having to visit the physician or a laboratory to have blood drawn. The exhaled breath concentrations would alert the physician when the drug dose needs to be adjusted.

It should be understood that the examples and embodiments described herein are for illustrative purposes only and that various modifications or changes in light thereof will be suggested to persons skilled in the art and are to be included within the spirit and purview of this application and the scope of the appended claims. Specifically, the marker detection method of the present invention is intended to cover detection not only through the exhalation by a patient with a device utilizing electronic nose technology, but also other suitable technologies, such as gas chromatography, transcutaneous/transdermal detection, semiconductive gas sensors, mass spectrometers, IR or UV or visible or fluorescence spectrophotometers.

All patents, patent applications, provisional applications, and publications referred to or cited herein, or from which a claim for benefit of priority has been made, are incorporated by reference in their entirety to the extent they are not inconsistent with the explicit teachings of this specification

1. A method of monitoring a patient during administration of at least one therapeutic drug, said method comprising:
administering to the patient at least one therapeutic drug;
exposing at least one sensor to expired gases from the patient;
detecting one or more target markers from the therapeutic drug with said sensor.

2. The method of claim 1 wherein said target marker is the therapeutic drug.

3. The method of claim 1 wherein said target marker is a metabolite of the therapeutic drug indicative of the therapeutic drug.

4. The method of claim 1 wherein said target marker is selected from a group consisting of dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene).

5. The method of claim 1 wherein at least one therapeutic drug is administered to the patient orally.

6. The method of claim 1 wherein at least one therapeutic drug is delivered intravenously.

7. The method of claim 1 wherein the detecting step comprises detecting both presence and concentration of the target marker to determine at least one therapeutic drug concentration in blood.

8. The method of claim 7 further comprising assigning a numerical value to the concentration as analyzed upon reaching a level of therapeutic effect of said therapeutic drug in said patient and, thereafter, assigning higher or lower values to the concentration based on its relative changes.

9. The method of claim 8, further comprising monitoring the concentration by monitoring changes in said value and adjusting administration of the therapeutic drug to maintain a desired therapeutic effect.

10. The method of claim 7 further comprising determining an appropriate dosage of at least one therapeutic drug based on the concentration of at least one target marker detected in said expired gases.

11. The method of claim 1 wherein the steps are repeated periodically to monitor pharmacodynamics and pharmacokinetics of at least one therapeutic drug over time.

12. The method of claim 1 wherein at least one therapeutic drug is for depression.

13. The method of claim 1 wherein at least one therapeutic drug is for analgesia.

14. The method of claim 1 wherein at least one therapeutic drug is selected for the treatment of a condition selected from group consisting of rheumatoid arthritis, systemic lupus erythematosus, angina, coronary artery disease, peripheral vascular disease, ulcerative colitis, Crohn's disease, organ rejection, epilepsy, anxiety, degenerative arthritis, vasculitis, and inflammation.

15. The method of claim 1 wherein the detecting is continuous.

16. The method of claim 1 wherein the detecting is periodic.

17. The method of claim 1 wherein at least one therapeutic drug is selected from the group consisting of: α-Hydroxy-Alprazolam; Acecainide (NAPA); Acetaminophen (Tylenol); Acetylmorphine; Acetylsalicylic Acid (as Salicylates); α-hydroxy-alprazolam; Alprazolam (Xanax); Amantadine (Symmetrel); Ambien (Zolpidem); Amikacin (Amikin); Amiodarone (Cordarone); Amitriptyline (Elavil) & Nortriptyline; Amobarbital (Amytal); Anafranil (Clomipramine) & Desmethylclomipramine; Ativan (Lorazepam); Aventyl (Nortriptyline); Benadryl (Dephenhydramine); Benziodiazepines; Benzoylecgonine; Benztropine (Cogentin); Bupivacaine (Marcaine); Bupropion (Wellbutrin) and Hydroxybupropion; Butabarbital (Butisol); Butalbital (Fiorinal) Carbamazepine (Tegretol); Cardizem (Diltiazem); Carisoprodol (Soma) & Meprobamate; and Celexa (Citalopram & Desmethylcitalopram).

18. The method of claim 1 wherein at least one therapeutic drug is selected from the group consisting of: Celontin (Methsuximide) (as desmethylmethsuximide); Centrax (Prazepam) (as Desmethyldiazepam); Chloramphenicol (Chloromycetin); Chlordiazepoxide; Chlorpromazine (Thorazine); Chlorpropamide (Diabinese); Clonazepam (Kionopin); Clorazepate (Tranxene); Clozapine; Cocaethylene; Codeine; Cogentin (Benztropine); Compazine (Prochlorperazine); Cordarone (Amiodarone); Coumadin (Warfarin); Cyclobenzaprine (Flexeril); Cyclosporine (Sandimmune); Cylert (Pemoline); Dalmane (Flurazepam) & Desalkylflurazepam; Darvocet; Darvon (Propoxyphene) & Norpropoxyphene; Demerol (Meperidine) & Normeperidine; Depakene (Valproic Acid); Depakote (Divalproex) (Measured as Valproic Acid); Desipramine (Norpramin); Desmethyldiazepam; Desyrel (Trazodone); Diazepam & Desmethyldiazepam; Diazepam (Valium) Desmethyldiazepam; Dieldrin; Digoxin (Lanoxin); Dilantin (Phenytoin); Disopyramide (Norpace); Dolophine (Methadone); Doriden (Glutethimide); Doxepin (Sinequan) and Desmethyldoxepin; Effexor (Venlafaxine); Ephedrine; Equanil (Meprobamate) Ethanol; Ethosuximide (Zarontin); Ethotoin (Peganone); Felbamate (Felbatol); Fentanyl (Innovar); Fioricet; Fipronil; Flunitrazepam (Rohypnol); Fluoxetine (Prozac) & Norfluoxetine; Fluphenazine (Prolixin); Fluvoxamine (Luvox); Gabapentin (Neurontin); Gamma-Hydroxybutyric Acid (GHB); Garamycin (Gentamicin); Gentamicin (Garamycin); Halazepam (Paxipam); Halcion (Triazolam); Haldol (Haloperidol); Hydrocodone (Hycodan); Hydroxyzine (Vistaril); Ibuprofen (Advil, Motrin, Nuprin, Rufen); Imipramine (Tofranil) and Desipramine; Inderal (Propranolol); Keppra (Levetiracetam); Ketamine; Lamotrigine (Lamictal); Lanoxin (Digoxin); Lidocaine (Xylocaine); Lindane (Gamma-BHC); Lithium; Lopressor (Metoprolol); Lorazepam (Ativan); and Ludiomil.

19. The method of claim 1 wherein at least one therapeutic drug is selected from the group consisting of: Maprotiline; Mebaral (Mephobarbital) & Phenobarbital; Mellaril (Thioridazine) & Mesoridazine; Mephenyloin (Mesantoin); Meprobamate (Miltown, Equanil); Mesantoin (Mephenyloin); Mesoridazine (Serentil); Methadone; Methotrexate (Mexate); Methsuximide (Celontin) (as desmethsuximide); Mexiletine (Mexitil); Midazolam (Versed); Mirtazapine (Remeron); Mogadone (Nitrazepam); Molindone (Moban); Morphine; Mysoline (Primidone) & Phenobarbital; NAPA & Procainamide (Pronestyl); NAPA (N-Acetyl-Procainamide); Navane (Thiothixene); Nebcin (Tobramycin); Nefazodone (Serzone); Nembutal (Pentobarbital); Nordiazepam; Olanzapine (Zyprexa); Opiates; Orinase (Tolbutamide); Oxazepam (Serax); Oxcarbazepine (Trileptal) as 10-Hydroxyoxcarbazepine; Oxycodone (Percodan); Oxymorphone (Numorphan); Pamelor (Nortriptyline); Paroxetine (Paxil); Paxil (Paroxetine); Paxipam (Halazepam); Peganone (Ethotoin); PEMA (Phenylethylmalonamide); Pentothal (Thiopental); Perphenazine (Trilafon); Phenergan (Promethazine); Phenothiazine; Phentermine; Phenylglyoxylic Acid; Procainamide (Pronestyl) & NAPA; Promazine (Sparine); Propafenone (Rythmol); Protriptyline (Vivactyl); Pseudoephedrine; Quetiapine (Seroquel); Restoril (Temazepam); Risperdal (Risperidone) and Hydroxyrisperidone; Secobarbital (Seconal); Sertraline (Zoloft) & Desmethylsertraline; Stelazine (Trifluoperazine); Surmontil (Trimipramine); Tocainide (Tonocard); and Topamax (Topiramate).

20. The method of claim 1 wherein said sensor is selected from the group consisting of: metal-insulator-metal ensemple (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, colorimetric tubes, infrared spectroscopy, gas chromatography, semiconductive gas sensor technology; mass spectrometers, gluorescent spectrophotometers, conductive polymer gas sensor technology; aptamer sensor technology; amplifying fluorescent polymer (AFP) sensor technology; or surface acoustic wave gas sensor technology.

21. The method of claim 20 wherein the sensor technology produces a unique electronic fingerprint to characterize the detection and concentration of said at least one target marker.

22. The method of claim 1 further comprising the step of recording data from said sensor.

23. The method of claim 1 further comprising the step of transmitting data from said sensor.

24. The method of claim 1 further comprising comparing at least one target marker detected with a predetermined signature profile.

25. The method of claim 1 further comprising capturing a sample of expired gases prior to exposing said sensor to expired gases.

26. The method of claim 1 further comprising dehumidifying expired gases prior to exposing said sensor to expired gases.

27. The method of claim 1 further comprising exposing said sensor to expired gases during exhalation of the patient's breath.

28. The method of claim 1 further comprising assigning a numerical value to the concentration as analyzed upon reaching a level of anesthetic effect in said patient and, thereafter, assigning higher or lower values to the concentration based on its relative changes.

29. The method of claim 1 wherein said sensor is portable.

30. A therapeutic drug delivery and monitoring system for delivering an appropriate dosage of the therapeutic drug to a patient:
at least one therapeutic drug supply having a controller for controlling the amount of therapeutic drug provided by the supply to the patient;
an expired gas sensor for analyzing the patient's breath for the presence and concentration of at least one target marker indicative of therapeutic drug concentrations in the patient's bloodstream, and for sending a signal regarding the concentration of the therapeutic drug in the patient's bloodstream; and
a system controller connected to the therapeutic drug supply, which receives and analyzes the signal from the sensor and controls the amount of therapeutic drug administered to the patient based on the signal.

31. The system of claim 30 wherein the expired gas sensor comprise a sensor for analyzing the gas for concentration of at least one target marker indicative of the therapeutic drug concentration in the patient's bloodstream and a processor for calculating the pharmacodynamic and pharmacokinetic effect of the therapeutic drug based on the concentration of the therapeutic drug.

32. The system of claim 31 wherein the sensor is selected from the group consisting of: metal-insulator-metal ensemple (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave (BAW) sensors, colorimetric tubes, infrared spectroscopy, gas chromatography, semiconductive gas sensor technology, mass spectrometers, gluorescent spectrophotometers, conductive polymer gas sensor technology; aptamer sensor technology; amplifying fluorescent polymer (AFP) sensor technology; or surface acoustic wave gas sensor technology.

33. The method of claim 1 wherein at least one therapeutic drug is a psychiatric drug.

34. The method, according to claim 33, wherein at least one therapeutic drug is selected from the group consisting of: antidepressants, anti-psychotics, anti-anxiety drugs, and depressants.
